(19) United States
(12) Patent Application Publication
(10) Pub. No.: US 2002/0183859 A1
(43) Pub. Date: Dec. 5, 2002

(54) Socket interface sleeve for a prosthetic device

(76) Inventor: Guy M. Houser, Bainbridge Island, WA (US)

Correspondence Address: Steven H. Arterberry, Esq., DORSEY & WHITNEY LLP, 1420 Fifth Avenue, Suite 3400, Seattle, WA, 98101, US

(21) Appl. No.: 09/874,533

(22) Filed: Jun. 4, 2001

(51) Int. Cl. A61F 2/78

(52) U.S. Cl. 623/36

(57) ABSTRACT

A socket interface sleeve is disclosed with a cylindrical member having an open end for receiving a limb, an opposing closed end, and a wall assembly comprised of a cushioning member formed from a polymeric gel. First and second fabric materials are disposed on the member to control support. The first material is elastically extensible in a first direction and is relatively elastically inextensible in a second direction, while the second material is extensible in the first direction and non-extensible in the second direction. In one embodiment, the first material is an elongated band extending the length of the sleeve. In a second embodiment, the first material extends from the midpoint to the end. In a third embodiment, the first material is uniformly elastic and extends from the opening to a midpoint of the sleeve.

### TECHNICAL FIELD

The present invention generally relates to prosthetic devices. More particularly, the invention relates to a socket interface member worn upon the residual limb of an amputee to provide a cushion between the residual limb and a prosthetic device.

### BACKGROUND OF THE INVENTION

Prosthesis sockets are generally rigid devices formed from relatively inflexible thermoplastics or fiber-reinforced thermoset materials that receive the residual limb of an amputee. Individuals requiring the use of prosthetic devices typically wear a socket interface sleeve over the residual limb to provide cushioning between the residual limb and the rigid prosthetic socket.

With reference to FIG. 1, a socket interface sleeve 1 for a residual limb 2 according to the prior art is shown. The socket interface sleeve 1 may include tubular socks comprised of cotton, wool or other synthetic fabrics, but more typically includes a relatively thick elastomeric cushioning member to provide additional comfort to the wearer. Once the wearer has inserted the residual limb 2 into the sleeve 1, the sleeve 1 is inserted into an interior space 3 of a prosthetic socket 4. The socket 4 then receives a docking member 5 that is attached to the sleeve 1 to lockably engage a mating docking member (not shown) attached to the socket 4 to secure the prosthetic socket 4 to the sleeve 1.

Although the use of elastomeric materials in the sleeve 1 significantly enhances the level of comfort achieved by the wearer, several drawbacks are associated with the use of elastomeric sleeves. For example, still referring to FIG. 1, some amputees may experience an effect generally referred to as “pistoning”, which is caused by the movement of the residual limb 2 relative to the sleeve 1 as the amputee ambulates. Movement of the residual limb 2 within the sleeve 1 may cause irritation to the residual limb 2. For example, localized irritations to the residual limb 2 such as abrasions or lesions may occur. In addition, the prolonged relative movement of the limb 2 in the sleeve 1 may also cause the sleeve 1 to prematurely wear out. Some amputees experience a significant contraction in the size of the residual limb 2 (atrophy) over a period of time, which may cause “pistoning” of the limb within the sleeve. Finally, partially enclosed air voids between the limb 2 and the sleeve 1 may be formed that periodically expand and contract during “pistoning”. During this periodic expansion and contraction of the air voids, air is expelled from the sleeve 1 during ambulation that generates undesirable sound effects. In some cases, the noise thus generated may cause embarrassment to the wearer. This effect further exacerbated by the presence of recessed areas on the residual limb 2, such as recesses that exist adjacent to the prominent tibia.

A further drawback associated with the sleeve 1, as shown in FIG. 1, is that the bony structure (not shown) in the residual limb 2 may locally compress regions in the sleeve 1 and elastically deform these regions such that the thickness of the elastomeric material is substantially reduced in these regions, resulting in a non-uniform fit and discomfort to the wearer. This effect may be particularly pronounced in cases where the residual limb still contains an articulating joint, as in below-the-knee, or below-the-elbow amputations. Other prior art methods have generally addressed the problem of local elastomer deformation in the sleeve 1 by augmenting the thickness of the cushioning material within the sleeve 1 in critical regions. For example, U.S. Pat. No. 5,830,237 to Kania addresses this problem by continuously adjusting the thickness of the cushioning material in the sleeve 1 to achieve an additional thickness of cushioning material in the critical regions. In an alternative approach, Kania discloses the addition of separately formed cushioning structures that are secured within the sleeve 1. A shortcoming inherent in this approach is that the varying thickness in the cushioning member is difficult and expensive to form by conventional methods, and the separately formed cushioning structures may become detached from the interior of the sleeve 1 during use.

Accordingly, there is a need in the art for a socket interface sleeve that resists elastomer deformation in critical regions, which may be fabricated easily and inexpensively.

### SUMMARY OF THE INVENTION

The invention is generally directed towards a socket interface sleeve for a use with a prosthetic device. In one aspect of the present invention, the sleeve is comprised of a cylindrical member with an open end for receiving a limb, an opposing closed end, and a wall assembly comprised of a cushioning member formed from a polymeric gel, and first and second fabric materials disposed on the polymeric gel. The first fabric material is relatively elastic in a first direction and is relatively inelastic in a second direction, while the second fabric layer is relatively inelastic in the first direction and is relatively elastic in the second direction. The first fabric material is formed in an elongated band and disposed on the sleeve to extend along the length of the sleeve to limit excessive circumferential stretching of the sleeve and is fixably joined to the second fabric material to comprise a unitary outer layer substantially covering the outer surface of the sleeve. The sleeve may also include a docking pad positioned on the closed end.

In another aspect of the invention, the first fabric material is disposed on the sleeve in an elongated band that extends from the closed end upwardly to approximately about the midpoint of the sleeve, and is fixably joined to the second fabric material to comprise a unitary outer layer substantially covering the outer surface of the wall assembly.

In still another aspect of the invention, the first fabric material is formed in an elongated band disposed on the sleeve that extends from the open end to approximately the midpoint of the wall assembly, and is uniformly elastic. The second fabric material is fixably joined to the first fabric material to comprise a unitary outer layer substantially covering the outer surface of the wall assembly.

### BRIEF DESCRIPTION OF THE DRAWINGS

FIG. 1 is a partial isometric view of a socket interface sleeve according to the prior art.

FIG. 2 is a frontal view of a socket interface sleeve according to an embodiment of the invention.

FIG. 3 is an isometric view of a socket interface sleeve according to an embodiment of the invention.

FIG. 4 is a partial cross sectional view of a socket interface sleeve according to an embodiment of the invention.

FIG. 5 is a further partial cross sectional view of a socket interface sleeve according to an embodiment of the invention that shows the material layers comprising the sleeve.

FIG. 6 is an isometric view of a docking pad for a socket interface sleeve according to an embodiment of the invention.

FIG 7 is a cross sectional view of a docking pad for a socket interface sleeve according to an embodiment of the invention.

FIG. 8 is a frontal view of a socket interface sleeve according to another embodiment of the invention.

FIG. 9 is an isometric view of a socket interface sleeve according to another embodiment of the invention.

FIG. 10 is a frontal view of a socket interface sleeve according to still another embodiment of the invention.

FIG. 11 is an isometric view of a socket interface sleeve according to still another embodiment of the invention.

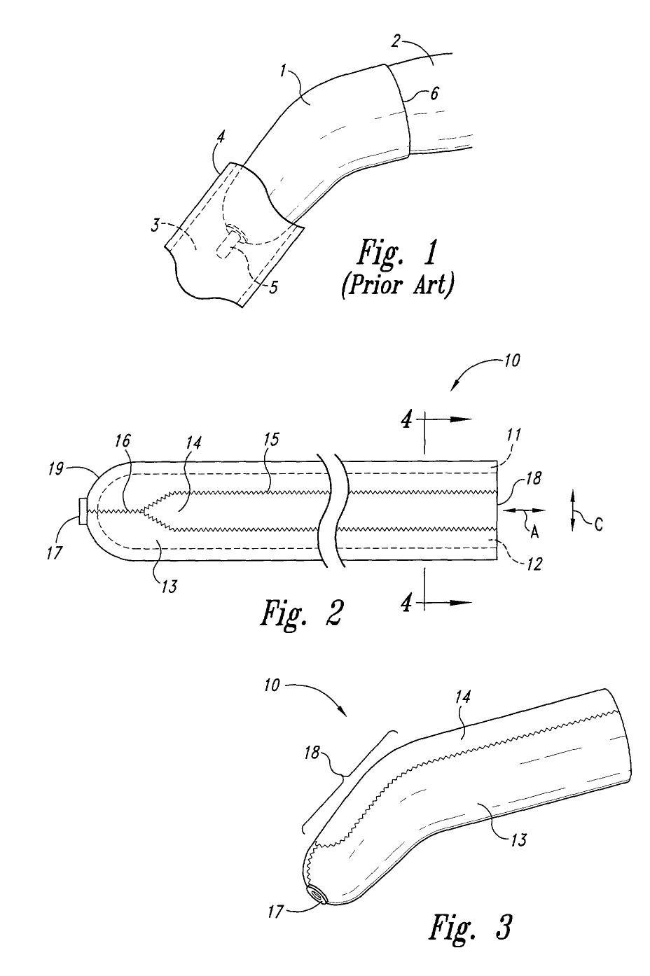

### DETAILED DESCRIPTION OF THE INVENTION

The present invention is generally directed to a socket interface member worn upon the residual limb of an amputee to provide a cushion between the residual limb and a prosthetic device. More particularly, the invention relates to a socket interface member having reinforcing fabric layers to support the residual limb, and to prevent the onset of the “pistoning” effect. Many of the specific details of certain embodiments of the invention are set forth in the following description and in FIGS. 2 through 11 to provide a thorough understanding of such embodiments. One skilled in the art will understand, however, that the present invention may have additional embodiments, or that the present invention may be practiced without several of the details described in the following description. Further, it is understood that the various embodiments of the socket interface sleeve disclosed herein may be equally applicable to interface sleeves worn by amputees where the residual limb is a portion of an arm or a portion of a leg.

A socket interface sleeve 10 according to an embodiment of the invention is shown in FIGS. 2 and 3. The sleeve 10 is a generally cylindrical member with an open end 18 for receiving a residual limb into an internal space 12, and an opposing closed end 19. A docking pad 17 is positioned on the closed end 19 to receive a docking pin (not shown). The docking pad 17 will be discussed in greater detail below. A wall assembly 11, which will also be described in more complete detail below, surrounds the internal space 12. A first fabric material 14 is disposed on a portion of the exterior surface of the wall assembly 11 that extends in an axial direction “A” from the open end 18 towards the closed end 19. A second fabric material 13 is disposed on a second portion of the wall assembly 11, which may cover the remaining portion of the wall assembly 11 not covered by the first fabric material 14. The first fabric material 14 is attached to the second fabric material 13 along a peripheral edge 15 of layer 14 to form a relatively unitary outer fabric layer on the wall assembly 11. The first fabric material 14 and second fabric material 13 may be formed by conventional means, such as cutting the layers 13 and 14 from a planar fabric material according to a pattern that will allow the layers 13 and 14 to conform to the generally cylindrical shape of the wall assembly 11. Accordingly, a seam 16 formed by joining edges of the fabric material 13 allows the fabric material 13 to surround the generally hemispherically shaped closed end 19. Alternatively, the fabric material 14 may extend all of the way to the end 19 so there is no seam 16 joining the opposite edges of the fabric material 13. The attachment of fabric material 13 to fabric material 14 along the peripheral edge 15, and the attachment at the seam 16 may be made by stitching the edges using a flat-locked stitch, which provides a relatively non-irritating seam. However, any type of thread and any type of stitch may be used. Alternatively, the attachment of fabric material 13 to fabric material 14 along the peripheral edge 15, and the attachment at the seam 16 may equivalently be made by thermally fusing the edges of the materials 13 and 14 together, by joining the edges using adhesives, by applying a thermally activated seam tape to the edges, or by still other means.

Still referring to FIGS. 2 and 3, the first fabric material 14 is comprised of a fabric that is anisotropically elastic so that it is inelastic in a direction “C”, which is approximately perpendicular to the axial direction “A”, and is relatively elastic in the direction “A”. The ratio of elastic extensibility is preferably at least 1:4. The second fabric material 13 is also anisotropically elastic, so that it is elastic in a direction “C”, which is approximately perpendicular to the axial direction “A”, and is relatively inelastic in the direction “A”. The materials used for the first fabric material 14 and the second fabric material 15 may be comprised of a segmented polyurethane fabric such as LYCRA®, or fabric blends comprised of LYCRA® and NYLON®. Still other equivalent fabric blends may be comprised additionally of CORDURA® and SUPPLEX®, all of which are available from E.I. Dupont de Nemours and Company of Wilmington, Del.

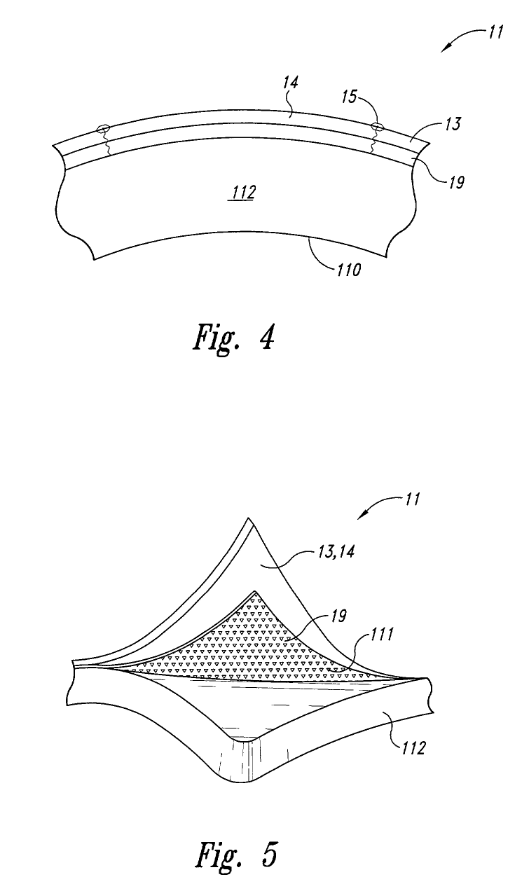

The wall assembly 11 of the sleeve 10 will now be described in greater detail, with reference to FIGS. 4 and 5. FIG. 4 shows a partial cross sectional view of the wall assembly 11. A cushioning member 112 is generally comprised of a continuous polymeric gel layer that is preferably at least about 0.12 inches in thickness. The polymeric gel used to form the cushioning member 112 may be comprised of a block copolymer mixed with a mineral oil. Suitable block copolymers include styrene isoprene/butadiene or styreneethylene/butadiene styrene block copolymers, although other block copolymers are equally suitable, and other resilient materials may also be used. For example, C-FLEX®, available from Consolidated Polymer Technologies of Largo, Fla., may be used. Alternatively, KRATON®, available from the Shell Chemical Company of Houston, Tex., or SEPTON®, available from Kurray Chemicals Co. of Japan may equivalently be used.

The cushioning member 112 has an interior surface 110 that forms an interface between the residual limb and the sleeve 10. The opposing surface of the member 112 may be attached to an intermediate fabric layer 19. Generally, the intermediate fabric layer is comprised of a tufted, or textured fabric having a bonding affinity to the materials used to fabricate the cushioning member 112. Accordingly, the intermediate fabric layer 19 may be comprised of a cotton terry cloth material, or other equivalent and suitably tufted fabrics. For example, a polyester fleece material, such as POLARTEC®, available from Malden Mills of Lawrence, Mass., may equivalently be used.

With reference now to FIG. 5, the tufted members 111 of the intermediate fabric layer 19 are more clearly shown, and generally become fixably mated to the member 112 during the molding operation used to fabricate the member 112 by projecting downwardly into the member 112 while member 112 is being formed to increase the bonding strength between the member 112 and the layer 19.

Returning now to FIG. 4, the fabric materials 13 and 14 are fixably bonded to the intermediate layer 19 and secured at the seam 15 to form a relatively flexible and resilient wall assembly 11. The intermediate fabric layer 19 is generally attached to a side of the fabric materials 13 and 14 prior to being positioned on the member 11 by adhesively fixing the layer 19 to the fabric materials 13 and 14. Although the intermediate fabric layer 19 may assist in bonding the fabric materials 13 and 14 to the cushioning member 112, it is understood that a socket interface sleeve not employing the intermediate fabric layer as described herein remains within the scope of the disclosed invention.

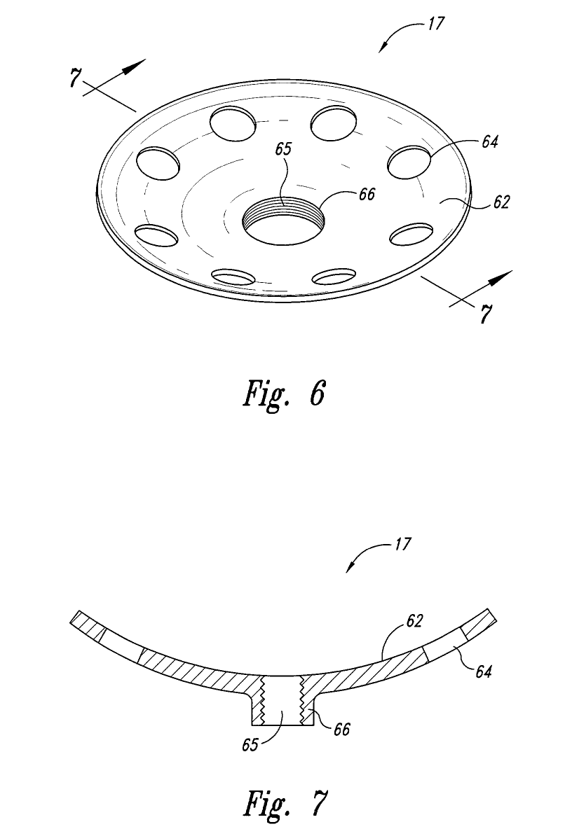

A docking pad 17 for the sleeve 10 will now be described with reference to FIGS. 6 and 7. The pad 17, as shown in FIG. 6, is a generally disk-shaped and inwardly concaved member 62 structured to conform to the generally hemispherical shape of the closed end 19 of sleeve 10 (as best seen in FIG. 2). The member 62 further includes a centrally positioned boss 66 that may be internally threaded, as shown, to receive a docking pin (not shown) or other means of attachment to the prosthetic device (not shown). The pad 17 may be adhesively affixed to the sleeve 10 by Room Temperature Vulcanizing (RTV) adhesives, or by various epoxy compounds. To facilitate improved bonding between the pad 17 and the closed end 19 of the sleeve 10, a plurality of circumferentially arranged holes 64 project through the member 62 to further enhance adhesive bonding between the pad 17 and the closed end 19. The docking member 17 may be formed from a metallic material, such as steel or aluminum, or a rigid polymer such as NYLON® or DELRIN® with a metallic threaded insert embedded at the boss 66.

Returning now to FIGS. 2 and 3, the limited elastic extensibility of the first fabric material 14 in the “C” direction advantageously provides sufficient support to the wall assembly 11 to prevent excessive thinning of the cushioning member 112 (as shown in FIGS. 4 and 5) in the region 18, as seen in FIG. 3. Excessive thinning of the cushioning member 112 beneath the material 14 may be caused by stretching of the cushioning member 112 in the circumferential direction “C” (see FIG. 2). By being relatively inelastic in the circumferential direction “C”, the fabric material 14 prevents the underlying cushioning member 112 from stretching and hence becoming thinner. The cushioning member 112 beneath the material 14 could also be thinned by other means were it not for the circumferential inelasticity of the fabric material 14. For example, by a tibial prominence within the residual limb contacting the cushioning member 112 as the amputee ambulates. The reduction in thinning resulting from the circumferential inelasticity of the fabric material 14 in the circumferential direction “C” advantageously reduces physical discomfort to the prosthetic user. Additionally, by supporting the sleeve 10 with fabric material 13 that is relatively inelastic in the axial direction “A”, as shown in FIG. 2, the occurrence of “pistoning” effects is reduced. Also, since the fabric material 14 extends over the knee of the amputee (if the amputation is below the knee), the sleeve 10 stretches easily over the knee as the knee is flexed. As a result, the sleeve 10 does not substantially restrict flexing of the knee.

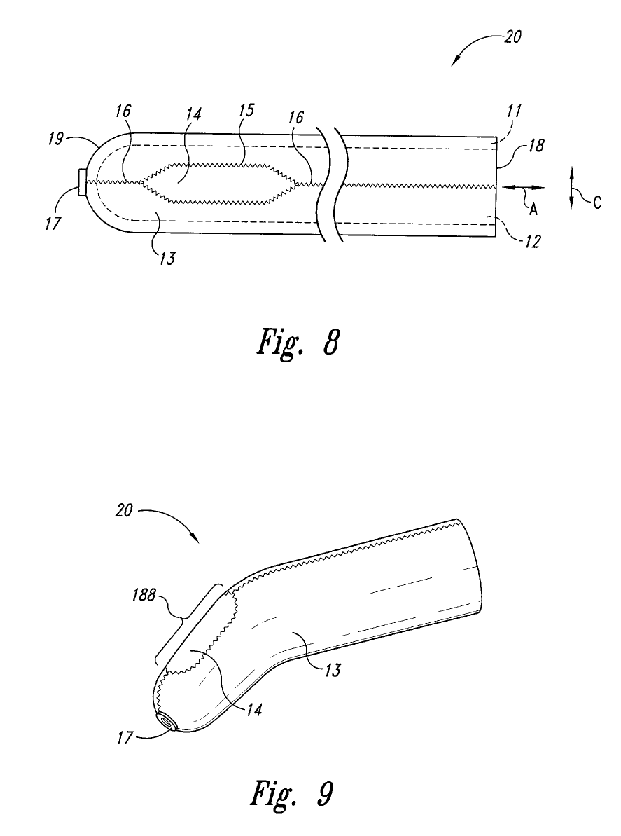

Turning now to FIG. 8, a frontal view of a socket interface sleeve 20 according to another embodiment of the invention is shown. As in the preceding embodiment, the sleeve 20 is a generally cylindrical member with an open end 18 for receiving a residual limb into an internal space 12, an opposing closed end 19, and a docking pad 17 positioned on the closed end 19. A wall assembly 11 similarly surrounds the internal space 12. The sleeve 20 has a first fabric material 14 disposed on a portion of the exterior surface of the wall assembly 11 that extends in an axial direction “A” from approximately about the closed end 19 upwardly towards the open end 18 to approximately the midpoint of the sleeve 20. A second fabric material 13 is disposed on a second portion of the wall assembly 11, which may cover the remaining portion of the wall assembly 11 not covered by the first fabric material 14. The first fabric material 14 is attached to the second fabric material 13 along a peripheral edge 15 of material 14 to form a relatively unitary outer fabric layer on the wall assembly 11.

Still referring to FIG. 8, the first fabric material 14 of the sleeve 20 is comprised of a fabric that is structured to be relatively inelastic in the circumferential direction “C”, and to be relatively elastic in the direction “A”, with the ratio of elastic extensibility again preferably being at least 1:4. The second fabric material 13 is again relatively elastic in the circumferential direction “C”, and to be relatively inelastic in the direction “A”, with the ratio of elastic extensibility again preferably being at least 1:4.

With reference now to FIGS. 8 and 9, the limited elastic extensibility of the first fabric material 14 in the “C” direction advantageously provides additional support to the wall assembly 11 in the region 188, as seen in FIG. 9. The sleeve 20 may provide sufficient support to amputees that do not require additional support above the sleeve midpoint.

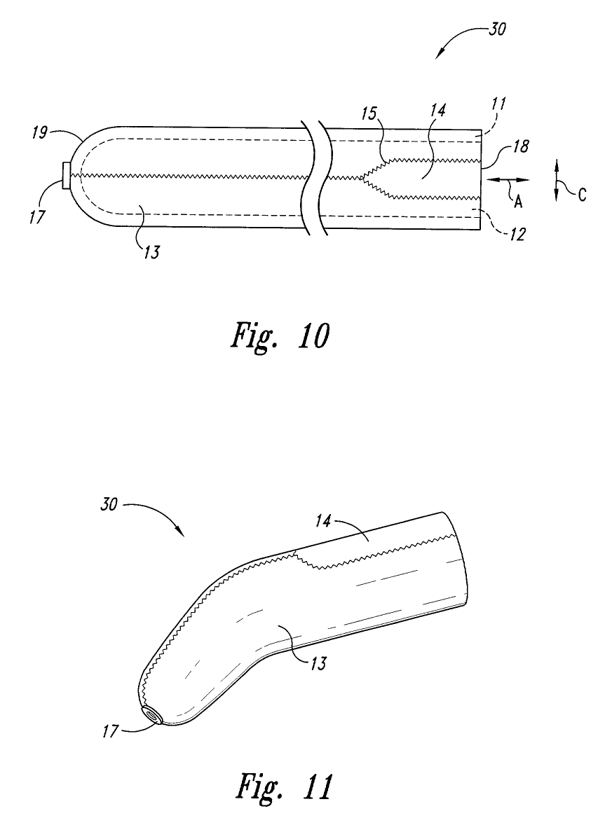

FIG. 10 is a frontal view of a socket interface sleeve 30 according to still another embodiment of the invention. As in the embodiments described above, the sleeve 30 is a generally cylindrical member with an open end 18 for receiving a residual limb into an internal space 12, and an opposing closed end 19 having a docking pad 17 positioned on the closed end 19. A wall assembly 11 surrounds the internal space 12. A first fabric material 14 is disposed on a portion of the exterior surface of the wall assembly 11 that extends in an axial direction “A” from the open end 18 downwardly towards the closed end 19 to an approximate midpoint location as shown. A second fabric material 13 is disposed on a second portion of the wall assembly 11, which may cover the remaining portion of the wall assembly 11 not covered by the first fabric material. The first fabric material 14 is attached to the second fabric material 13 along a peripheral edge 15 of material 14 to form a relatively unitary outer fabric layer on the wall assembly 11.

With reference to FIGS. 10 and 11, the first fabric material 14 is comprised of a fabric that is isotropically elastic, so that it is uniformly extensible in all directions, while the second fabric material 13 is structured to be relatively inelastic in a direction “A”, and to be elastic in the direction “C”, with the ratio of elastic extensibility also preferably being at least 1:4. This embodiment may significantly reduce the discomfort experienced by some wearers who require the additional overall support offered by a relatively large fabric material that is relatively inelastic in the “A” direction, as shown in FIG. 10. In addition, by providing a uniformly elastic material 14 on the sleeve 30 at a position above the approximate position of the knee, additional flexibility is attained by amputees with a residual limb that includes a knee joint.

The description of the illustrated embodiments of the invention is not intended to be exhaustive or to limit the invention to the precise form disclosed. While specific embodiments of, and examples of, the invention are described in the foregoing for illustrative purposes, various equivalent modifications are possible within the scope of the invention, as those skilled in the relevant art will recognize. Moreover, the various embodiments described above may be combined to provide further embodiments. Accordingly, the invention is not limited by the disclosure, but instead the scope of the invention is to be determined entirely by the following claims.

1. A sleeve for enclosing a residual limb, comprising:
a generally tubular member having an axial length and an exterior surface, an open end for receiving the residual limb and an opposing closed end;
a first material positioned in a first region of the tubular member, the first material being elastic in a first direction, and relatively inelastic in a second direction that is approximately perpendicular to the first direction; and
a second material positioned in a second region of the tubular member, the second material being relatively inelastic in the first direction, and elastic in the second direction, wherein the first and second materials substantially enclose the member.

2. The sleeve according to claim 1 wherein the first and second materials are further comprised of a fabric continuously bonded to the exterior surface of the tubular member.

3. The sleeve according to claim 2 wherein the first material is comprised of an elongated fabric band having a peripheral edge, the band extending axially along the exterior surface of the member from the open end to at least approximately the closed end, the first direction being approximately aligned with the axial length, and the second material is comprised of an elongated fabric band that extends axially and circumferentially across the second region, the second material being attached to the first material along the peripheral edge.

4. The sleeve according to claim 3 wherein the second material is attached to the first material by stitching the second material to the first material along the peripheral edge.

5. The sleeve according to claim 3 wherein the second material is attached to the first material by adhesively bonding the second material to the first material along the peripheral edge.

6. The sleeve according to claim 3 wherein the second material is attached to the first material by thermally joining the second material to the first material along the peripheral edge using a thermally activated seam tape.

7. The sleeve according to claim 3 wherein the second material is attached to the first material by thermally joining the second material to the first material along the peripheral edge using an ultrasonic or a thermal weld.

8. The sleeve according to claim 2 wherein the first material is comprised of an elongated fabric band having a peripheral edge, the band extending axially along the exterior surface of the member from at least approximately the midpoint to approximately the closed end, the first direction being approximately aligned with the axial length, and the second material is comprised of an elongated fabric band that extends axially and circumferentially across the second portion, the second material being attached to the first material along the peripheral edge.

9. The sleeve according to claim 8 wherein the second material is attached to the first material by stitching the second material to the first material along the peripheral edge.

10. The sleeve according to claim 8 wherein the second material is attached to the first material by adhesively bonding the second material to the first material along the peripheral edge.

11. The sleeve according to claim 8 wherein the second material is attached to the first material by thermally joining the second material to the first material along the peripheral edge using a thermally activated seam tape.

12. The sleeve according to claim 8 wherein the second material is attached to the first material by thermally joining the second material to the first material along the peripheral edge using a ultrasonic or a thermal weld.

13. The sleeve according to claim 1 wherein the first material and the second material are further comprised of a back surface, the back surface having an intermediate fabric layer continuously attached thereon that is structured to bond to the exterior surface of the tubular member.

14. The sleeve according to claim 13 wherein the intermediate fabric layer is further comprised of a fabric with surface tufting having a high bonding affinity with the tubular member.

15. The sleeve according to claim 14 wherein the intermediate fabric layer is comprised of a cotton terry cloth.

16. The sleeve according to claim 14 wherein the intermediate fabric layer is comprised of a polyester fleece material.

17. The sleeve according to claim 16 wherein the intermediate fabric layer comprises POLARTEC®.

18. The sleeve according to claim 2 wherein the first and second materials are comprised of a segmented polyurethane fabric.

19. The sleeve according to claim 18 wherein the segmented polyurethane fabric comprises LYCRA®.

20. The sleeve according to claim 18 wherein the first and second materials are further comprised of a fabric blend comprising LYCRA® and NYLON®.

21. The sleeve according to claim 20 wherein the fabric blend is further comprised of CORDURA®.

22. The sleeve according to claim 21 wherein the fabric blend is further comprised of SUPPLEX®.

23. The sleeve according to claim 21 wherein the fabric blend is further comprised of approximately about 30% CORDURA®, approximately about 60% NYLON® and approximately about 10% LYCRA®.

24. The sleeve according to claim 1 wherein the tubular member is further comprised of a polymeric gel.

25. The sleeve according to claim 24 wherein the polymeric gel is further comprised of a block copolymer mixed with a mineral oil.

26. The sleeve according to claim 25 wherein the block copolymer is further comprised of a styrene isoprene/butadiene block copolymer.

27. The sleeve according to claim 25 wherein the block copolymer is further comprised of a styrene ethylene/butadiene styrene block copolymer.

28. The sleeve according to claim 25 wherein the block copolymer is further comprised of C-FLEX®.

29. The sleeve according to claim 25 wherein the block copolymer is further comprised of KRATON®.

30. The sleeve according to claim 25 wherein the block copolymer is further comprised of SEPTON®.

31. The sleeve according to claim 1 further comprising a docking pad positioned on the closed end of the tubular member.

32. The sleeve according to claim 31 wherein the docking pad is adhesively joined to the tubular member.

33. The sleeve according to claim 31 wherein the docking pad is comprised of a metal.

34. The sleeve according to claim 31 wherein the docking pad is comprised of a polymeric material.

35. A sleeve for enclosing a residual limb, comprising:
a generally tubular member having an axial length and an exterior surface, an open end for receiving the residual limb and an opposing closed end;
a first material positioned in a first region of the tubular member, the first material being relatively uniformly elastic in a first and a second direction, the second direction being approximately perpendicular to the first direction; and
a second material positioned in a second region of the tubular member, the second material being elastic in the second direction, and relatively inelastic in the first direction, wherein the first and second materials substantially enclose the member.

36. The sleeve according to claim 35 wherein the first and second materials are further comprised of a fabric continuously bonded to the exterior surface of the tubular member.

37. The sleeve according to claim 36 wherein the first material is comprised of an elongated fabric band having a peripheral edge, the band extending axially along the exterior surface of the member from the open end to at least approximately a midpoint of the axial length, and the second material is comprised of an elongated fabric band that extends axially and circumferentially across the second region with the first direction being approximately aligned with the axial length, and the first material being attached to the second material along the peripheral edge.

38. The sleeve according to claim 37 wherein the second material is attached to the first material by stitching the second material to the first material along the peripheral edge.

39. The sleeve according to claim 37 wherein the second material is attached to the first material by adhesively bonding the second material to the first material along the peripheral edge.

40. The sleeve according to claim 37 wherein the second material is attached to the first material by thermally joining the second material to the first material along the peripheral edge using a thermally activated seam tape.

41. The sleeve according to claim 37 wherein the second material is attached to the first material by thermally joining the second material to the first material along the peripheral edge using an ultrasonic or a thermal weld.

42. The sleeve according to claim 35 wherein the first material and the second material are further comprised of a back surface, the back surface having an intermediate fabric layer continuously attached thereon that is structured to bond to the exterior surface of the tubular member.

43. The sleeve according to claim 42 wherein the intermediate fabric layer is further comprised of a fabric with surface tufting having a high bonding affinity with the tubular member.

44. The sleeve according to claim 43 wherein the intermediate fabric layer is comprised of a cotton terry cloth.

45. The sleeve according to claim 43 wherein the intermediate fabric layer is comprised of a polyester fleece material.

46. The sleeve according to claim 45 wherein the intermediate fabric layer comprises POLARTEC®.

47. The sleeve according to claim 36 wherein the first and second materials are comprised of a segmented polyurethane fabric.

48. The sleeve according to claim 47 wherein the segmented polyurethane fabric comprises LYCRA®.

49. The sleeve according to claim 48 wherein the first and second materials are further comprised of a fabric blend comprising LYCRA® and NYLON®.

50. The sleeve according to claim 49 wherein the fabric blend is further comprised of CORDURA®.

51. The sleeve according to claim 50 wherein the fabric blend is further comprised of SUPPLEX®.

52. The sleeve according to claim 50 wherein the fabric blend is further comprised of approximately about 30% CORDURA®, approximately about 60% NYLON® and approximately about 10% LYCRA®.

53. The sleeve according to claim 35 wherein the tubular member is further comprised of a polymeric gel.

54. The sleeve according to claim 53 wherein the polymeric gel is further comprised of a block copolymer mixed with a mineral oil.

55. The sleeve according to claim 54 wherein the block copolymer is further comprised of a styrene isoprene/butadiene block copolymer.

56. The sleeve according to claim 54 wherein the block copolymer is further comprised of a styrene ethylene/butadiene styrene block copolymer.

57. The sleeve according to claim 54 wherein the block copolymer is further comprised of C-FLEX®.

58. The sleeve according to claim 54 wherein the block copolymer is further comprised of KRATON®.

59. The sleeve according to claim 54 wherein the block copolymer is further comprised of SEPTON®.

60. The sleeve according to claim 35 further comprising a docking pad positioned on the closed end of the tubular member.

61. The sleeve according to claim 60 wherein the docking pad is adhesively joined to the tubular member.

62. The sleeve according to claim 60 wherein the docking pad is comprised of a metal.

63. The sleeve according to claim 60 wherein the docking pad is comprised of a polymeric material.

64. A sleeve for enclosing a residual limb, the sleeve having a generally cylindrical shape with an interior space, an exterior area, and an axial length with an opening at one end of the axial length to receive the residual limb into the interior space and a closed end at the opposing end of the axial length, the sleeve further comprising:
a first elastic region that is elastically stretchable in a first direction, and relatively non stretchable in a second direction, the first direction being approximately perpendicular to the second direction; and
a second elastic region that is relatively non-elastically stretchable in the first direction and elastically stretchable in the second direction, wherein the first and second elastic regions substantially enclose the interior space.

65. The sleeve according to claim 64 wherein the first elastic region is at least about four times more elastically stretchable in the first direction than in the second direction.

66. The sleeve according to claim 64 wherein the first elastic region is at least about as elastically stretchable in the first direction as the second elastic region in the second direction.

67. The sleeve according to claim 64 wherein the first elastic region and the second elastic region are comprised of a fabric material disposed on the exterior area of the sleeve.

68. The sleeve according to claim 67 wherein the first elastic region is comprised of an elongated fabric band having a peripheral edge, the band extending in the axial direction along the exterior surface from the opening to approximately about the closed end with the first direction being substantially aligned with the axial length, and the second elastic region is comprised of an elongated fabric band that extends axially and circumferentially across the exterior surface, the first region being joined to the second region along the peripheral edge by a fabric attachment.

69. The sleeve according to claim 68 wherein the fabric attachment comprises a flat interlocking stitch along the peripheral edge.

70. The sleeve according to claim 68 wherein the fabric attachment comprises adhesively bonding the first region to the second region.

71. The sleeve according to claim 68 wherein the fabric attachment comprises thermally joining the first region to the second region along the peripheral edge using a thermally activated seam tape.

72. The sleeve according to claim 68 wherein the fabric attachment comprises thermally joining the second material to the first material along the peripheral edge using an ultrasonic weld or a thermal weld.

73. The sleeve according to claim 67 wherein the first elastic region is comprised of an elongated fabric band having a peripheral edge, the band extending in the axial direction along the exterior area from approximately about the midpoint of the axial length to approximately about the closed end with the first direction being substantially aligned with the axial length, and the second region is comprised of an elongated fabric band that extends axially and circumferentially across the exterior area, the first region being joined to the second region along the peripheral edge by a fabric attachment.

74. The sleeve according to claim 73 wherein the fabric attachment comprises a flat interlocking stitch along the peripheral edge.

75. The sleeve according to claim 73 wherein the fabric attachment comprises adhesively bonding the first region to the second region.

76. The sleeve according to claim 73 wherein the fabric attachment comprises thermally joining the first region to the second region along the peripheral edge using a thermally activated seam tape.

77. The sleeve according to claim 73 wherein the fabric attachment comprises thermally joining the first region to the second region along the peripheral edge using an ultrasonic weld or a thermal weld.

78. The sleeve according to claim 67 wherein the first elastic region and the second elastic region are further comprised of a back surface having an intermediate fabric layer disposed thereon.

79. The sleeve according to claim 78 wherein the intermediate fabric layer is comprised of a cotton terry cloth.

80. The sleeve according to claim 78 wherein the intermediate fabric layer is comprised of a polyester fleece material.

81. The sleeve according to claim 80 wherein the intermediate fabric layer comprises POLARTEC®.

82. The sleeve according to claim 64 wherein the first elastic layer and the second elastic layer are comprised of a segmented polyurethane fabric.

83. The sleeve according to claim 82 wherein the segmented polyurethane fabric comprises of LYCRA®.

84. The sleeve according to claim 82 wherein the first elastic layer and the second elastic layer are comprised of a fabric blend comprising LYCRA® and NYLON®.

85. The sleeve according to claim 84 wherein the fabric blend is further comprised of CORDURA®.

86. The sleeve according to claim 84 wherein the fabric blend is further comprised of SUPPLEX®.

87. The sleeve according to claim 85 wherein the fabric blend is further comprised of at least about 30% CORDURA®, at least about 60% NYLON and at least about 10% LYCRA.

88. The sleeve according to claim 64, further comprising an elastomeric cushioning member.

89. The sleeve according to claim 88 wherein the elastomeric cushioning member is comprised of a block copolymer mixed with a mineral oil.

90. The sleeve according to claim 89 wherein the block copolymer is further comprised of a styrene isoprene/butadiene block copolymer.

91. The sleeve according to claim 89 wherein the block copolymer is further comprised of a styreneethylene/butadiene styrene block copolymer.

92. The sleeve according to claim 89 wherein the block copolymer is further comprised of C-FLEX®.

93. The sleeve according to claim 89 wherein the block copolymer is further comprised of KRATON®.

94. The sleeve according to claim 89 wherein the block copolymer is further comprised of SEPTON®.

95. A sleeve for enclosing a residual limb, the sleeve having a generally cylindrical shape with an interior space, an exterior area, and an axial length with an opening at one end of the axial length to receive the residual limb into the interior space and a closed end at the opposing end of the axial length, the sleeve further comprising:
a first elastic region that is uniformly elastically stretchable in a first direction and a second direction, the first direction being approximately perpendicular to the second direction; and
a second elastic region that is elastically stretchable in the second direction and relatively inelastic in the first direction, wherein the first and second elastic regions substantially enclose the interior space.

96. The sleeve according to claim 95 wherein the first elastic region is at least about four times more elastically stretchable in the first direction than in the second direction.

97. The sleeve according to claim 95 wherein the first elastic region is at least about as elastically stretchable in the first and second directions as the second elastic region in the second direction.

98. The sleeve according to claim 95 wherein the first elastic region and the second elastic region are comprised of a fabric material disposed on the exterior area of the sleeve.

99. The sleeve according to claim 98 wherein the first elastic region is comprised of an elongated fabric band having a peripheral edge, the band extending in the axial direction along the exterior area from the opening to approximately about the midpoint of the axial length, and the second elastic region is comprised of an elongated fabric band that extends axially and circumferentially across the exterior area with the first direction being substantially aligned with the axial length, and the first region being joined to the second region along the peripheral edge by a fabric attachment.

100. The sleeve according to claim 99 wherein the fabric attachment comprises a flat interlocking stitch along the peripheral edge.

101. The sleeve according to claim 99 wherein the fabric attachment comprises adhesively bonding the first region to the second region.

102. The sleeve according to claim 99 wherein the fabric attachment comprises thermally joining the first region to the second region along the peripheral edge using a thermally activated seam tape.

103. The sleeve according to claim 99 wherein the fabric attachment comprises thermally joining the first region to the second region along the peripheral edge using an ultrasonic weld or a thermal weld.

104. The sleeve according to claim 95 wherein the first elastic region and the second elastic region are further comprised of a back surface having an intermediate fabric layer disposed thereon.

105. The sleeve according to claim 104 wherein the intermediate fabric layer is comprised of a cotton terry cloth.

106. The sleeve according to claim 104 wherein the intermediate fabric layer is comprised of a polyester fleece material.

107. The sleeve according to claim 95 wherein the intermediate fabric layer comprises POLARTEC®.

108. The sleeve according to claim 95 wherein the first elastic layer and the second elastic layer are comprised of a segmented polyurethane fabric.

109. The sleeve according to claim 108 wherein the segmented polyurethane fabric comprises of LYCRA®.

110. The sleeve according to claim 109 wherein the first elastic layer and the second elastic layer are comprised of a fabric blend comprising LYCRA® and NYLON®.

111. The sleeve according to claim 110 wherein the fabric blend is further comprised of CORDURA®.

112. The sleeve according to claim 108 wherein the fabric blend is further comprised of SUPPLEX®.

113. The sleeve according to claim 112 wherein the fabric blend is further comprised of at least about 30% CORDURA®, at least about 60% NYLON and at least about 10% LYCRA.

114. The sleeve according to claim 95, further comprising an elastomeric cushioning member.

115. The sleeve according to claim 114 wherein the elastomeric cushioning member is comprised of a block copolymer mixed with a mineral oil.

116. The sleeve according to claim 115 wherein the block copolymer is further comprised of a styrene isoprene/butadiene block copolymer.

117. The sleeve according to claim 115 wherein the block copolymer is further comprised of a styreneethylene/butadiene styrene block copolymer.

118. The sleeve according to claim 115 wherein the block copolymer is further comprised of C-FLEX®.

119. The sleeve according to claim 115 wherein the block copolymer is further comprised of KRATON®.

120. The sleeve according to claim 115 wherein the block copolymer is further comprised of SEPTON®.

121. A method for cushioning the limb of an amputee, comprising:
supporting a first portion of the limb with a first elastic member that is elastically stretchable in a first direction approximately parallel to a lateral axis of the limb, and relatively non stretchable in a second direction that is approximately perpendicular to the first direction; and
supporting a second portion of the limb with a second elastic member that is relatively non-elastically stretchable in the first direction and elastically stretchable the second direction.

122. The method according to claim 121 wherein the act of supporting a first portion of the limb with a first elastic member is further comprised of supporting an anterior portion of the limb.

123. The method according to claim 122 wherein the act of supporting an anterior portion of the limb further comprises supporting a portion of the limb from an anterior proximal portion of the limb to the anterior distal end of the limb.

124. The method according to claim 122 wherein the act of supporting an anterior portion of the limb further comprises supporting a portion of the limb from an anterior medial portion of the limb to the anterior distal end of the limb.

125. A method for cushioning the limb of an amputee, comprising:
supporting a first portion of the limb with a first elastic member that is uniformly elastically stretchable in a first direction and a second direction, the first direction being approximately perpendicular to the second direction and the first direction being approximately parallel to a lateral axis of the limb; and
supporting a second portion of the limb with a second elastic member that is relatively non-elastically stretchable in the first direction and elastically stretchable the second direction.

126. The method according to claim 125 wherein the act of supporting a first portion of the limb with a first elastic member is further comprised of supporting an anterior portion of the limb.

127. The method according to claim 126 wherein the act of supporting an anterior portion of the limb further comprises supporting a portion of the limb from an anterior proximal portion of the limb to the anterior medial portion of the limb.
